(19) United States
(12) Patent Application Publication
(10) Pub. No.: US 2014/0219942 A1
(43) Pub. Date: Aug. 7, 2014

(54) SUNSCREENS

(71) Applicant: Christine MENDROK-EDINGER, Basel (CH)

(72) Inventor: Christine MENDROK-EDINGER, Basel (CH)

(73) Assignee: DSM IP ASSETS B.V., Heerlen (NL)

(21) Appl. No.: 14/124,679

(22) PCT Filed: Jun. 6, 2012
(86) PCT No.: PCT/EP2012/060664

§ 371 Date: Mar. 10, 2014

(30) Foreign Application Priority Data
Jun. 8, 2011 (EP) 11169198.6
Jun. 15, 2011 (EP) 11169921.1
Jul. 12, 2011 (EP) 11173611.2

(51) Int. Cl. A61K 8/49; A61K 8/58; A61K 8/34; A61Q 5/00; A61K 8/42; A61K 8/37; A61Q 19/00

(52) U.S. Cl. CPC A61K 8/496; A61K 8/37; A61K 8/58; A61Q 19/00; A61Q 5/00; A61K 8/42; A61K 8/342
USPC 424/70.1

(57) ABSTRACT

The present invention relates to a composition for topical application comprising at least a benzotriazol derivative and a cosmetic solvent selected from the group consisting of benzoate solvents such as phenethyl benzoate, dimethyl capramide and diethylhexyl 2,6-naphthalate. Furthermore, the invention relates to the use of specific benzotriazol derivatives to mask the off-odor of cosmetic solvents such as particularly phenethyl benzoate, dimethyl capramide and diethylhexyl 2,6-naphthalate.

The present invention relates to a composition for topical application comprising at least a benzotriazol derivative and a cosmetic solvent selected from the group consisting of benzoate solvents such as phenethyl benzoate, dimethyl capramide and diethylhexyl 2,6-naphthalate. Furthermore, the invention relates to the use of specific benzotriazol derivatives to mask the off-odor of cosmetic solvents such as particularly phenethyl benzoate, dimethyl capramide and diethylhexyl 2,6-naphthalate.

Cosmetic solvents such as phenethyl benzoate, dimethyl capramide or diethylhexyl 2,6-naphthalate often exhibit excellent solubilisation properties for solid UV-filter substance such as avobenzone and oxybenzone. However, many cosmetic solvents have a significant off-odor which is maintained after incorporation into topical compositions which is highly undesirable.

Thus, there is an ongoing need for substances which are able to mask the unpleasant smell of cosmetic solvents such as particularly of phenethyl benzoate, dimethyl capramide or diethylhexyl 2,6-naphthalate in topical compositions.

Surprisingly, it has been found that the use of specific benzotriazol derivatives in topical compositions is able to mask the unpleasant smell (off-odor) of cosmetic solvents such as e.g. phenethyl benzoate, dimethyl capramide or diethylhexyl 2,6-naphthalate.

Thus, the invention relates to the use of a benzotriazol derivative of formula (I)

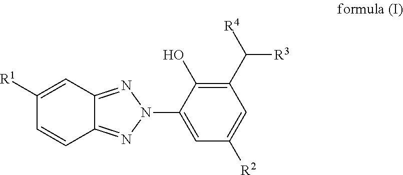

wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen.
for masking the off-odor of cosmetic solvents such as in particular of phenethyl benzoate, dimethyl capramide or diethylhexyl 2,6-naphthalate. In particular the invention relates to the use of a benzotriazol derivative of formula (I) for masking the off-odor of phenethyl benzoate, dimethyl capramide or diethylhexyl 2,6-naphthalate such as particularly of phenethyl benzoate in a topical composition. Such compositions are novel.

Thus, in another aspect, the invention also relates to a topical composition comprising at least one benzotriazol derivative of formula (I) and a cosmetic solvent selected from the group consisting of benzoate solvents such as particularly phenethyl benzoate, dimethyl capramide and diethylhexyl 2,6-naphthalate.

In yet a further embodiment the invention relates to a method of masking the off-odor of cosmetic solvents such as in particular cosmetic solvents selected from the group consisting of benzoate solvents such as particularly phenethyl benzoate, dimethyl capramide and diethylhexyl 2,6-naphthalate, said method comprising the addition of at least one benzotriazol derivative of formula (I) into said topical composition and observing or appreciating the result. In a particular embodiment, the cosmetic solvent is selected from the group of benzoate solvents and even more in particular the cosmetic solvent is phenethyl benzoate.

The term “C_{x-y}alkyl” as used herein refers to straight-chain or branched alkyl radicals having x to y carbon atoms such as e.g. methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethyl propyl, 1-methylpentyl, 2-methyl pentyl, 3-methylpentyl, 4-methylpentyl, 1,1-dimethyl propyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl without being limited thereto.

The term C₅₋₁₀cycloalkyl denotes to unsubstituted or C₁₋₁₀alkyl (mono- or poly-)substituted, in particular C₁₋₅alkyl (mono- or poly-)substituted cyclic, bicyclic or tricyclic hydrocarbon residues such as in particular cyclopentyl, cyclohexyl, cycloheptyl or decahydronaphtyl. Preferably, C₅₋₁₀cycloalkyl denotes to unsubstituted or C₁₋₂alkyl (mono- or poly-)substituted cyclopentyl, cyclohexyl or cycloheptyl such as in particular to unsubstituted or (mono- or poly-)methyl substituted cyclohexyl such as most in particular cyclohexyl or 3,3,5-trimethyl-cyclohexyl.

The term “C_{x-y}alkoxy” as used herein denotes to linear or branched alkoxy-, respectively unsubstituted or (mono- or poly-)substituted cycloalkoxy radicals having from x to y carbon atoms such as e.g. methoxy, ethoxy, propoxy, butyloxy or pentyloxy, 2,5,5-trimethylhexyloxy, 3,5,5-trimethylhexyloxy, isoamyloxy, 2-ethylhexyloxy or 3,3,5-trimethyl-cyclohexyloxy.

The term C₆₋₁₀aryl refers e.g. to naphthyl or phenyl radicals, preferably phenyl.

The term “cosmetic solvent” as used herein relates to any solvent conventionally used in topical, in particular topical cosmetic compositions. The cosmetic solvents have the functional use to dissolve other ingredients such as in particular solid ingredients, e.g. solid UV-filter substances such as e.g. butyl methoxy dibenzoylmethane. Furthermore the cosmetic solvents are liquid at ambient temperature (25° C.)

The term “benzoate solvent” refers in particular to Behenyl Benzoate (Finsolv 137), Benzyl Benzoate (Morflex Benzyl Benzoate), Butyloctyl Benzoate, C12-15 Alkyl Benzoate (Finsolv TN), C16-17 Alkyl Benzoate (Finsolv G2), Castor Oil Benzoate (Finsolv BCO-115), Cetyl Ricinoleate Benzoate (Finsolv BCR-111), C12-15 Pareth-3 Benzoate (Dermol Z5-3B), Dipentaerythrityl Hexabehenate/Benzoate/Ethylhexanoate (Nomcort HR-822), Dipropylene Glycol Dibenzoate (Finsolv PG-22, Uniflex 9-88), Ethylhexyl Benzoate (Finsolv EB), Ethylhexyl Hydroxystearate Benzoate (Finsolv BOHS-111), Hexyldecyl benzoate, Isobutyl Benzoate, Isostearyl Benzoate (Finsolv SB), laureth-2 Benzoate (Berne) Ester 126), Lauryl/Myristyl Benzoate (Corum 5014), Linalyl Benzoate (Linalyl Benzoate), Octyldodecyl Benzoate (Finsolv BOD), Oleth-2 Benzoate, Panthenyl Ethyl Ether Benzoate, Pentaerythrityl Tetrabehenate/Benzoate/Ethyl hexanoate (Salacos P-B822), Pentaerythrityl Tetrabenzoate (Uniplex 552), Pentaerythrityl Tetraethylhexanoate/Benzoate (Salacos BO-63, Salacos P-B8(75)), Phenethyl Benzoate (X-Tend 226, Finsolv Sun), Phenyl Benzoate, PPG-15 Stearyl Ether Benzoate (Finsolv P), Propyl Benzoate, Propylene Glycol Dibenzoate (LexFeel Shine), Stearyl Benzoate (Finsolv 116).

Phenethyl benzoate [INCI Name] is also known as benzoic acid, 2-phenyl ethyl ester [CAS No. 94-47-3] and is e.g. available as X-Tend™226 at International Specialty Products (I.S.P.).

Dimethyl capramide [INCI Name] is also known as N,N-dimethyldecanamide [CAS No. 14433-76-2] and is e.g. commercially available at Hallstar as Spectrasolv DMDA.

Diethylhexyl 2,6-naphthalate [INCI Name] is also known as 6-naphthalenedicarboxylic acid, 2,6-bis(2-ethylhexyl) ester [CAS No. 127474-91-3] and is e.g. commercially available at Hallstar as Corapan TQ.

The cosmetic solvent is preferably used in an amount of 0.5 to 20 wt.-% such as in particular of 1 to 10 wt.-% such as most in particular of 1 to 6 wt.-%, based on the total weight of the composition.

The benzoate solvent such as in particular phenethyl benzoate is preferably used in an amount of 0.5 to 20 wt.-% such as in particular of 1 to 10 wt.-% such as most in particular of 1 to 6 wt.-%, based on the total weight of the composition.

The amount of the at least one benzotriazol derivative of formula (I) in the compositions according to the invention is not critical and at least 0.01 wt.-%. Preferable the amount of the at least one benzotriazol derivative of formula (I) in the compositions according to the invention is selected in the range of 2 to 20 wt.-%, such as in the range of 2 to 15 wt.-%, in particular in the range of 4 to 10 wt.-%, and most particular in the range of 4 to 8 wt.-% based on the total weight of the composition.

In a particular embodiment of the present invention the benzotriazol derivative is selected from compounds of formula (I) wherein R¹ and R⁴ are hydrogen, R² is methyl and R³ is C₅₋₁₀alkoxy such as preferably C₆₋₁₀alkoxy, or C₆cycloalkoxy such as in particular 2,5,5-trimethylhexyloxy, 3,5,5-trimethylhexyloxy, isoamyloxy, 2-ethylhexyloxy or 3,3,5-trimethyl-cyclohexyloxy. Such compounds and their preparation are e.g. disclosed in EP Application No.: 10150832.3 (PCT publication: WO2011/086124). Particularly preferred according to the present invention is a compound of formula (I), wherein R¹ and R⁴ are hydrogen, R² is methyl and R³ is 2-ethylhexyloxy (i.e. 2-(2H-Benzotriazol-2-yl)-6-(2-ethylhexyloxymethyl)-4-methyl-phenol).

In another particular embodiment of the present invention the compound of formula (I) is a compound wherein R¹ and R⁴ are hydrogen, R² is methyl and R³ is undecyl (C₁₁H₂₃) which is commercially available as Tinogard TL [INCI Name: benzotriazolyl dodecyl p-cresol; IUPAC Name: 2-(2H-benzotriazol-2-yl)-6-dodecyl-4-methyl-phenol] at BASF SE Ludwigshafen.

In another particular embodiment the compositions according to the present invention are substantially free of a polyglycerol based UV-filter such as e.g. disclosed in [EP Application No's] EP09178503.0, EP09178501.4, EP09178502.2 EP09178495.9, EP09178506.3, EP09178505.5 or EP10150832.3 which are obtainable by a process comprising the steps of ring-opening polymerization of x mol equivalents of glycidol using 1 mol equivalent of a polyol starter unit with y mol equivalents hydroxyl-groups, followed by block copolymerization with z×(x+y) mole equivalents of propylene oxide to form a hyperbranched polyether-polyol backbone carrying (x+y) mol equivalents hydroxyl-groups followed by partial or total esterification, respectively partial or total etherification of the hydroxyl groups with a UV-light absorbing chromophore such as particularly with p-dimethylamino benzoic acid, 3-[1-(4-Hydroxymethyl-phenyl)-meth-(E)-ylidene]-1,7,7-trimethyl-bicyclo[2.2.1]heptan-2-one, 2-(4-diethylamino-2-hydroxybenzoyl)benzoic acid, p-alkoxycinnamic acid, 2-cyano-3,3-diphenylacrylic acid as well as mixtures thereof and wherein x is an integer selected in the range from 3-16, y is an integer selected in the range from 1-6, and z is an integer selected in the range from 0-10.

The term “topical” is understood here to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, and keratinous fibres. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations, hair care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples for care preparations are hair-washing preparations in the form of shampoos, hair conditioners, hair-care preparations such as e.g. pretreatment preparations, hair tonics, styling creams, gels such as styling gels, pomades, hair rinses, treatment packs, intensive hair treatments, hair-straightening preparations, liquid hair-setting preparations, hair foams (hair mousses) and hairsprays.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are light-protective preparations, such as sun protection milks, sun protection lotions, sun protection creams, sun protection oils, sun blocks or tropical's or day care creams with a SPF (sun protection factor). Of particular interest are sun protection creams, sun protection lotions, sun protection milks and sun protection preparations.

In another particular embodiment the topical compositions are hair-washing preparations in the form of shampoos or hair treatment preparations intended to be left in the hair (and not washed out) such as hair-setting preparations, hairsprays, gels, pomades, styling creams or hair foams (hair mousses), particularly hairsprays, gels or hair foams (hair mousses).

A shampoo may, for example, have the following composition: from 0.01 to 5 wt.-% of benzotriazol derivative of formula (I), 0.5 to 5 wt.-% of phenethyl benzoate, 12.0 wt.-% of sodium laureth-2-sulfate, 4.0 wt.-% of cocamidopropyl betaine, 3.0 wt.-% of sodium chloride, and water ad 100 wt.-%.

The topical compositions according to the present invention may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si—)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

The topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples.

If the topical composition according to the invention is an O/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, lsosteareth-20, Ceteareth-20, Oleth-20, Steareth-20, Steareth-21, Ceteth-20, Isoceteth-20, Laureth-23, Steareth-100, glycerylstearatcitrate, glycerylstearate (self emulsifying), stearic acid, salts of stearic acid, polyglyceryl-3-methylglycosedistearate. Further suitable emulsifiers are phosphate esters and the salts thereof such as cetyl phosphate (Amphisol® A), diethanolamine cetyl phosphate (Amphisol® DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one O/W respectively Si/W emulsifier is preferably used in an amount of 0.5 to 10 wt.-% such as in particular in the range of 0.5 to 5 wt.-% such as most in particular in the range of 1 to 4 wt.-% based on the total weight of the composition.

Particular suitable O/W emulsifiers according to the present invention encompass phosphate esters emulsifier of formula (II)

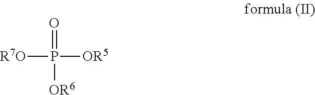

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. Particular advantageous phosphate ester emulsifiers according to the invention are cetyl phosphates such as in particular potassium cetyl phosphate e.g. commercially available as Amphisol® K at DSM Nutritional Products Ltd Kaiseraugst.

Further suitable O/W emulsifiers are polyethyleneglycol (PEG) esters or diesters such as e.g. [INCI Names] (Glyceryl Stearate (and) PEG-100 Stearate), PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate. Particularly preferred according to the present invention is PEG-100 Stearate sold under the tradename Arlacel™ 165 (INCI Glyceryl Stearate (and) PEG-100 Stearate) by Croda.

Another particular suitable class of O/W emulsifiers are non ionic self-emulsifying systems derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

In particular embodiment, the invention relates to topical compositions according to the present invention which are in the form of O/W emulsions comprising an oily phase dispersed in an aqueous phase in the presence of an O/W emulsifier wherein the benzotriazol derivative of formula (I) is benzotriazolyl dodecyl p-cresol and the O/W emulsifier is selected from the group of cetyl phosphates such as particularly potassium cetyl phosphate.

In another particular embodiment, the invention relates to topical compositions according to the present invention which are in the form of O/W emulsions comprising an oily phase dispersed in an aqueous phase in the presence of an O/W emulsifier wherein the benzotriazol derivative of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy, such as in particular 2-(2H-Benzotriazol-2-yl)-6-(2-ethylhexyloxymethyl)-4-methyl-phenol) and the O/W emulsifier is selected from the group of cetyl phosphates such as particularly potassium cetyl phosphate. The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16) cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

The compositions in form of O/W emulsions according to the invention can be provided, for example, in all the formulation forms for O/W emulsions, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

Finally, a subject-matter of the invention is a method for the cosmetic treatment of keratinous substances such as in particular the skin, characterized in that a composition as defined above is applied to the said keratinous substances such as in particular to the skin. The method is in particular suitable to protect the skin against the adverse effects of UV-radiation such as in particular sun-burn and/or photoageing.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

Suitable UV-filter substances may be organic or inorganic compounds. Exemplary organic UV-filter substances encompass e.g. acrylates such as e.g. 2-ethylhexyl 2-cyano-3,3-diphenylacrylate (octocrylene, PARSOL® 340), ethyl 2-cyano-3,3-diphenylacrylate; Camphor derivatives such as e.g. 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, terephthalylidene dicamphor sulfonic acid (Mexoryl® SX); Cinnamate derivatives such as e.g. ethylhexyl methoxycinnamate (PARSOL® MCX), ethoxyethyl methoxycinnamate, isoamyl methoxycinnamate as well as cinnamic acid derivatives bond to siloxanes; p-Aminobenzoic acid derivatives such as e.g. p-aminobenzoic acid, 2-ethylhexyl p-dimethylaminobenzoate, N-oxypropylenated ethyl p-aminobenzoate, glyceryl p-aminobenzoate; Benzophenones such as e.g. benzophenone-3, benzophenone-4,2,2′,4,4′-tetrahydroxy-benzophenone, 2,2′-dihydroxy-4,4′-dimethoxybenzophenone; Esters of benzalmalonic acid such as e.g. di-(2-ethylhexyl) 4-methoxybenzalmalonate; Organosiloxane compounds carrying chromophore groups such as e.g. polysilicones-15 (PARSOL® SLX), drometrizole trisiloxane (Mexoryl® XL); Imidazole derivatives such as e.g. 2-phenyl benzimidazole sulfonic acid (PARSOL®HS) and salts thereof such as e.g. sodium- or potassium salts, ammonium salts, morpholine salts, salts of primary, sec. and tert. amines like monoethanolamine salts, diethanolamine salts; Salicylate derivatives such as e.g. isopropylbenzyl salicylate, benzyl salicylate, butyl salicylate, ethylhexyl salicylate (PARSOL® EHS, Neo Heliopan® OS), isooctyl salicylate or homomethyl salicylate (homosalate, PARSOL® HMS, Neo Heliopan® HMS); Triazine derivatives such as e.g. ethylhexyl triazone (Uvinul® T-150), diethylhexyl butamido triazone (Uvasorb® HEB), bis-ethylhexyloxyphenol methoxyphenyl triazine (Tinosorb® 5); Benzotriazole derivatives such as e.g. 2,2′-methylene-bis-(6-(2H-benzotriazole-2-yl)-4-(1,1,3,3-tetramethylbutyl)-phenol (Tinosorb® M); Encapsulated UV-filters such as e.g. encapsulated ethylhexyl methoxycinnamate (Eusolex® UV-pearls) or microcapsules loaded with UV-filters as e.g. disclosed in EP 1471995; Phenylene-1,4-bis-benzimidazolsulfonic acids or salts such as e.g. 2,2-(1,4-phenylene)bis-(1H-benzimidazol-4,6-disulfonic acid) (Neoheliopan AP); Benzoxazol-derivatives such as e.g. 2,4-bis-[5-1(dimethylpropyl)benzoxazol-2-yl-(4-phenyl)-imino]-6-(2-ethylhexyl)-imino-1,3,5-triazin [Uvasorb® K2A); Dibenzoylmethane derivatives such as e.g. Butyl Methoxy Dibenzoylmethane; IUPAC Name: 1-(4-Methoxyphenyl)-3-(4-tert-butylphenyl)propane-1,3-dione) which is e.g. commercially available as PARSOL® 1789 at DSM Nutritional Products Ltd and amino substituted benzophenones such as Diethylamino hydroxybenzoyl hexyl benzoate [IUPAC Name: Hexyl 2-[4-(diethylamino)-2-hydroxybenzoyl]benzoate] sold under the tradename Uvinul® A plus by BASF.

Inorganic UV-filter substances encompass pigments such as e.g. microparticulated Zink oxide or Titanium dioxide (e.g. commercially available as PARSOL® TX) The term “microparticulated” refers to a particle size from about 5 nm to about 200 nm, particularly from about 15 nm to about 100 nm. The particles may also be coated by other metal oxides such as e.g. aluminum or zirconium oxides or by silica or by organic coatings such as e.g. polyols, methicone, aluminum stearate, alkyl silane. Such coatings are well known in the art.

In order to enhance the photostability of sun care products it may be desirable to add a photostabilizer. Exemplary photostabilizers known to a skilled person in the art encompass e.g. 3,3-diphenylacrylate derivatives such as e.g. octocrylene (PARSOL® 340) or Polyester-8 (Polycrylene®) or Methoxycrylene (Solastay S1®); Benzylidene camphor derivatives such as e.g. 4-methyl benzylidene camphor (PARSOL® 5000); Benzalmalonate derivatives such as e.g. polysilicones-15 (PARSOL® SLX) or diethylhexyl syringylidene malonate (Oxynex ST liquid); Dialkyl naphthalates such as diethylhexyl naphthalate (Corapan TQ) without being limited thereto. An overview on further stabilizers is e.g. given in ‘SPF Boosters & Photostability of Ultraviolet Filters’, HAPPI, October 2007, p. 77-83 which is included herein by reference. The photostabilizers are generally used in an amount of 0.05 to 10 wt.-% with respect to the total weigh of the topical composition.

Generally, the amount of each UV-filter substance in the compositions according to the invention is selected in the range of about 0.1 to 10 wt.-%, preferably in the range of about 0.2 to 7 wt.-%, most preferably in the range of about 0.5 to 5 wt.-% with respect to the total weigh of the topical composition.

The total amount of UV-filter substances in the compositions according to the invention is preferably in the range of about 1 to 40 wt.-%, preferably in the range of about 5 to 30 wt.-%, in particular in the range of 10 to 30 wt.-% with respect to the total weight of the topical composition.

Preferred UVB-filter substances according to the invention encompass polysilicones-15, phenylbenzimidazol sulfonic acid, octocrylene, ethylhexyl methoxycinnamate, ethylhexyl triazone, ethyl hexylsalicylate, 4-methyl benzylidene camphor, benzophenones-3 and/or homosalate.

Preferred broadband UV-filter substances according to the invention encompass unsymmetrical s-triazine derivatives such 2,4-Bis-{[4-(2-ethyl-hexyloxy)-2-hydroxy]-phenyl}-6-(4-methoxyphenyl)-1,3,5-triazin, certain benzophenones such as e.g. 2-Hydroxy-4-methoxy-benzophenon, 2,2′-Methylen-bis-(6-(2H-benzotriazol-2-yl)-4-(1,1,3,3-tetramethyl-butyl)-phenol), and/or titanium dioxide.

Preferred UVA-filter substances encompass Butyl Methoxy Dibenzoylmethane and Diethylamino hydroxybenzoyl hexyl benzoate.

It is particularly suitable, if the topical compositions according to the present invention are sun care preparations comprising at least one, preferably at least two further UV-filter substances. Preferably the additional UV filter substances are selected from the group consisting of butyl methoxydibenzoylmethane, octocrylene, 4-methylbenzylidene camphor, homomethyl salicylate and microfine titanium dioxide such as PARSOL TX as well as mixtures thereof. Most preferably butyl methoxydibenzoylmethane, octocrylene, 4-methylbenzylidene camphor, homomethyl salicylate and microfine titanium dioxide such as PARSOL TX are present. Preferably, butyl methoxydibenzoylmethane is used in an amount ranging from 1 to 5 wt.-%, 4-methylbenzylidene camphor in an amount of 1 to 5 wt.-%, octocrylene in an amount of 2 to 10 wt and homomethyl salicylate in an amount of 1 to 10 wt.-% and titanium dioxide in an amount of 1 to 5 wt.-%, based on the total weight of the composition. Preferably the sun care preparations are O/W emulsions comprising an oily phase dispersed in an aqueous phase in the presence of an O/W emulsifier and the O/W emulsifier is potassium cetylphosphate, preferably used in an amount ranging from 1 to 5 wt.-%.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992), The Cosmetic, Toiletry and Fragrance Association, Inc. without being limited thereto.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH, Triethanolamine (TEA Care), Tromethamine (Trizma Base) and Aminomethyl Propanol (AMP-Ultra PC 2000) according to standard methods in the art.

The following examples are provided to further illustrate the compositions and effects of the present invention. These examples are illustrative only and are not intended to limit the scope of the invention in any way.

### EXAMPLE

The following formulations have been prepared according to standard methods. Afterwards the odor of the samples was assessed by a sensory panel of 6 persons by opening the flask and smelling at the flask. The off odor intensity of the respective formulation was rated at a scale from 1 (strong smell) to 3 (no smell). The indicated values are mean values of the 6 panelists.

The odor of the pure compounds was rated as follows:

| | |
|---|---|
| Phenethyl Benzoate | 1 |
| Benzotriazolyl Dodecyl p-Cresol | 3 |
| 2-(2H-Benzo-triazol-2-yl)-6-(2-ethylhexyloxy-methyl)-4-methyl- | 3 |
| phenol | |
| Dicaprylyl carbonate | 3 |

As can be retrieved from the table below the sample containing the benzotriazol derivatives had almost no smell, whereas the sample containing phenethyl benzoate exhibited a significant off-odor. Furthermore, it can be retrieved that the benzotriazol derivatives lead to a significant decrease of the off odor of phenethyl benzoate.

| Trade Name | INCI Name | Wt.-% | Wt.-% | Wt.-% |
|---|---|---|---|---|
| PARSOL ® 1789 | Butyl Methoxydibenzoylmethane | 4.5 | 4.5 | 4.5 |
| PARSOL ® 340 | Octocrylene | 8 | 8 | 8 |
| AMPHISOL K | Potassium Cetyl Phosphate | 2 | 2 | 2 |
| Lanette ® O | Cetearyl Alcohol | 1.5 | 1.5 | 1.5 |
| Mixed Tocopherol 95 | Tocopherol | 0.2 | 0.2 | 0.2 |
| Antaron V-216 | VP/Hexadecene Copolymer | 1 | 1 | 1 |
| PARSOL ® 5000 | 4-Methylbenzylidene Camphor | 3 | 3 | 3 |
| PARSOL ® HMS | Homosalate | 5 | 5 | 5 |
| PARSOL ® TX | Titantium Dioxide & Dimethicone & Silica | 3 | 3 | 3 |
| X-Tend 226 | Phenethyl Benzoate | | 5 | 5 |
| Tinogard ® TL | Benzotriazolyl Dodecyl p-Cresol | 5 | 5 | — |
| Cetiol ® CC | Dicaprylyl Carbonate | 5 | — | 5 |
| 1,3-Butylenglykol | Butylene Glycol | 3 | 3 | 3 |
| Edeta BD | Disodium EDTA | 0.1 | 0.1 | 0.1 |
| Pemulen ® TR-1 | Acrylates/C10-30 Alkyl Acrylate Crosspolymer | 0.15 | 0.15 | 0.15 |
| Water dem. | Aqua | Ad 100 | Ad 100 | Ad 100 |
| Keltrol ® CG SF | Xanthan Gum | 0.15 | 0.15 | 0.15 |
| Trizma Base 25% solution | Tromethamine | 1.3 | 1.3 | 1.3 |
| Ethanol | Alcohol | 6 | 6 | 6 |
| Odor | | 2.8 | 1.8 | 1.3 |
| PARSOL ® 1789 | Butyl Methoxydibenzoylmethane | 4.5 | 4.5 | 4.5 |
| PARSOL ® 340 | Octocrylene | 8 | 8 | 8 |
| AMPHISOL ® K | Potassium Cetyl Phosphate | 2 | 2 | 2 |
| Lanette ® O | Cetearyl Alcohol | 1.5 | 1.5 | 1.5 |
| Mixed Tocopherol 95 | Tocopherol | 0.2 | 0.2 | 0.2 |
| Antaron V-216 | VP/Hexadecene Copolymer | 1 | 1 | 1 |
| PARSOL ® 5000 | 4-Methylbenzylidene Camphor | 3 | 3 | 3 |
| PARSOL ® HMS | Homosalate | 5 | 5 | 5 |
| PARSOL ® TX | Titantium Dioxide & Dimethicone & Silica | 3 | 3 | 3 |
| X-Tend 226 | Phenethyl Benzoate | | 5 | 5 |
| | 2-(2H-Benzo-triazol-2-yl)-6-(2-ethylhexyl-oxy- | 5 | 5 | — |
| | methyl)-4-methyl-phenol* | | | |
| Cetiol CC | Dicaprylyl Carbonate | 5 | — | 5 |
| 1,3-Butylenglykol | Butylene Glycol | 3 | 3 | 3 |
| Edeta BD | Disodium EDTA | 0.1 | 0.1 | 0.1 |
| Pemulen TR-1 | Acrylates/C10-30 Alkyl Acrylate Crosspolymer | 0.15 | 0.15 | 0.15 |
| Water dem. | Aqua | Ad 100 | Ad 100 | Ad 100 |
| Keltrol CG SF | Xanthan Gum | 0.15 | 0.15 | 0.15 |
| Trizma Base 25% solution | Tromethamine | 1.3 | 1.3 | 1.3 |
| Ethanol | Alcohol | 6 | 6 | 6 |
| Odor | | 2.8 | 2.2 | 1 |

*benzotriazol derivative of formula (I): 2-(2H-Benzotriazol-2-yl)-6-(2-ethylhexyloxymethyl)-4-methyl-phenol (i.e. a compound of formula (I), wherein R¹ and R⁴ are hydrogen, R² is methyl and R³ is 2-ethylhexyloxy)

1. A topical composition comprising a cosmetic solvent selected from the group consisting of benzoate solvents, dimethyl capramide and diethylhexyl 2,6-naphthalate and at least one benzotriazol derivative of formula (I) 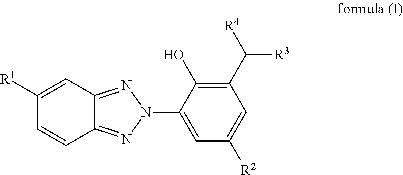
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen.

2. The topical compositions according to claim 1, characterized in that the benzoate solvent is phenethyl benzoate.

3. The topical composition according to claim 1, characterized in that the amount of phenethyl benzoate is selected in the range of 0.5 to 20 wt.-%, based on the total weight of the composition.

4. The topical composition according to claim 1, characterized in that the benzotriazol derivative is used in an amount selected in the range of 2 to 20 wt.-% based on the total weight of the composition.

5. The topical composition according to claim 1, characterized in that the benzotriazol compound of formula (I) is a compound wherein R¹ and R⁴ are hydrogen, R² is methyl and R³ is 2,5,5-trimethylhexyloxy, 3,5,5-trimethylhexyloxy, isoamyloxy, 2-ethylhexyloxy, 3,3,5-trimethyl-cyclohexyloxy or undecyl.

6. The topical composition according to claim 1, characterized in that the topical composition is an O/W emulsion comprising an oily phase dispersed in an aqueous phase in the presence of an O/W emulsifier.

7. The topical composition according to claim 6, characterized in that the O/W emulsifier is selected from the group consisting of phosphate esters.

8. The topical composition according to claim 7, characterized in that the O/W emulsifier is potassium cetyl phosphate.

9. The topical composition according to claim 6, characterized in that the amount of O/W emulsifier is selected in the range of 0.5 to 10 wt.-%, based on the total weight of the composition.

10. The topical composition according to claim 1, characterized in that the composition comprises at least one co-surfactant in an amount selected in the range of 0.1 to 10 wt.-% based on the total weight of the composition.

11. The topical composition according to claim 10, characterized in that the co-surfactant is selected from the group consisting of cetyl alcohol, cetearyl alcohol, stearyl alcohol, behenyl alcohol, glyceryl stearate, glyceryl myristate and hydrogenated coco-glycerides as well as mixtures thereof.

12. The topical composition according to claim 1, characterized in that the topical composition is a skin care preparation, hair care preparation, decorative preparation or a functional preparation.

13. Use of a benzotriazol derivative of formula (I) 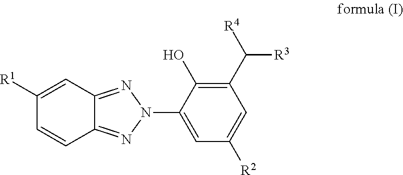
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl; C₅₋₁₀cycloalkyl; C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl; preferably hydrogen
for masking the off-odor of cosmetic solvents.

14. Method of masking the off-odor of cosmetic solvents, said method comprising the addition of at least one benzotriazol derivative of formula (I) 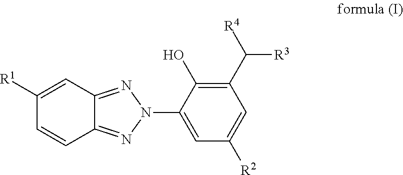
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloalkyl; C₁₋₂₀alkoxy or C₅₋₁₀cycloalkoxy; preferably C₅₋₁₅alkyl or C₅₋₁₅alkoxy; and
R⁴ is hydrogen or C₁₋₅alkyl; preferably hydrogen
into said topical composition and observing or appreciating the result.

15. The use or method according to claim 13, characterized in that the cosmetic solvent is phenethyl benzoate.

16. A method for the cosmetic treatment of keratinous substances, characterized in that a composition as defined in claim 1 is applied to the said keratinous substances.
